(19) United States
(12) Patent Application Publication
(10) Pub. No.: US 2022/0305720 A1
(43) Pub. Date: Sep. 29, 2022

(54) INFECTION-RESISTANT AND BIOACTIVE INTERBODY DEVICE, AND ASSOCIATED COMPOSTION AND METHOD

(71) Applicant: Cleveland State University, Cleveland, OH (US)

(72) Inventor: Prabaha Sikder, Cleveland, OH (US)

(73) Assignee: Cleveland State University, Cleveland, OH (US)

(21) Appl. No.: 17/706,997

(22) Filed: Mar. 29, 2022

Related U.S. Application Data
(60) Provisional application No. 63/200,777, filed on Mar. 29, 2021.

(51) Int. Cl. B29C 64/118; C08K 3/32; C08K 9/02; A61F 2/44; B33Y 70/10; B33Y 80/00; A61F 2/30

(52) U.S. Cl. CPC B29C 64/118; C08K 3/32; C08K 9/02; A61F 2/4455; B33Y 70/10; B33Y 80/00; A61F 2/30942; B29K 2071/00

(57) ABSTRACT

An article (e.g., an interbody device) contains a polymer (e.g., polyetheretherketone (PEEK)) and transition metal-doped amorphous magnesium phosphate.

The present application claims priority to U.S. provisional patent application Ser. No. 63/200,777, filed Mar. 29, 2021, which is incorporated by reference herein in its entirety.

### BACKGROUND

Improper functioning of fusion cages may lead to a spinal fusion procedure that might not be completely successful in alleviating a patient's back or neck pain. The improper functioning may result from the incorrect choice in the makeup material of the interbody cage.

For instance, polyetheretherketone (PEEK) is a bioinert material (i.e., it cannot bind with neighboring bone). This can lead to poor fixation of the implant and no new bone regeneration at the intervertebral space.

In addition, PEEK is prone to bacterial colonization which increases infection incidences. The state-of-the-art involves the usage of antibiotics which has the following shortcomings. First, repeated usage of antibiotics helps in the mutation of antibiotic-resistant bacteria which then causes intractable infections. Second, the application of antibiotics is systemic in nature and it does not promise a localized treatment thus increasing therapeutic time. Third, antibiotics can cause side-effects to various patients. Fourth, treatment costs are increased.

At present, there are no PEEK interbody cages that bind with bone rapidly and inhibit bacterial colonization. Thus, there is a critical need to develop multi-functional cage materials; devoid of which cages will often fail to perform and reduce success rates of fusion surgeries.

State-of-the-art interbody cages utilize additional coatings which have the following shortcomings. First, the coatings can delaminate from the implant surface. Second, the corroded coating particles can have an adverse effect on the intervertebral microenvironment. Third, coatings increase the cost of the interbody cage.

It would be desirable to develop new interbody devices that overcome the problems associated with PEEK and coatings.

### BRIEF DESCRIPTION

The present technology involves the symbiotic combination of a polymer (e.g., PEEK) and a novel multi-functional material known as magnesium phosphate. The result is an infection-resistant and bioactive interbody device which will be the first-of-its-kind in the spine market. The technology involves the localized release of ions to resist infections and stimulate bone formation. Importantly, for the first time, the interbody cages are developed by a sustainable additive manufacturing technique which helps in significantly reducing product cost.

Disclosed, in some embodiments, is an interbody device. The device comprises polyetheretherketone (PEEK) and magnesium phosphate.

Disclosed, in other embodiments, is a process for forming an article (e.g., product, device, implant, etc.) comprising PEEK and magnesium phosphate via additive manufacturing.

Disclosed, in further embodiments, is a composition comprising PEEK and magnesium phosphate.

Also disclosed are articles containing a polymer and a transition metal-doped amorphous magnesium phosphate.

The article may be an interbody device. Non-limiting examples include internal bone fixation devices, bioactive and regenerative constructs, and synthetic bone scaffolds or plates.

The magnesium phosphate is doped with at least one transition metal. Non-limiting examples include silver, copper, and zinc.

The article may be formed via additive manufacturing. In particular embodiments, the additive manufacturing is fused filament fabrication.

The polymer may be a polyaryletherketone. Non-limiting examples include polyetheretherketone (PEEK) and polyetherketoneketone (PEKK).

The article may contain compositions that comprise about 5-30 vol % of the magnesium phosphate, including from about 12-25 vol % and about 15-20 vol %.

The transition metal-doped amorphous magnesium phosphate may contain about 5-20 wt % of transition metal(s), based on the weight of magnesium, including about 8-15 wt % and about 6-12 wt %.

Further disclosed are processes for forming a composite article. The processes include extruding a mixture of magnesium phosphate and a polymer to form composite filaments; and fabricating the article from the extruded composite filaments via additive manufacturing.

The process may further include combining magnesium phosphate and a polymer and mixing in a power mixer prior to the extrusion.

Extrusion may be performed with heater temperatures in the range of about 340° C. to about 360° C.

Composite filaments are also disclosed. The filaments contain a polymer; and magnesium phosphate.

Processes for patient-specific implant design and production are disclosed and generally include acquiring at least one image from a patient; processing the image to design a patient-specific implant; and forming the patient-specific implant. The patient-specific implant comprises a polymer and a transition metal-doped amorphous magnesium phosphate.

In some embodiments, the processes further include implanting the patient-specific implant in the patient.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a flow chart illustrating a method for forming an article in accordance with some embodiments of the present disclosure.

FIG. 2 is a flow chart illustrating a patient-specific treatment method in accordance with some embodiments of the present disclosure.

FIG. 3 is a perspective view of an interbody device in accordance with some embodiments of the present disclosure.

FIG. 4 is a photograph of a prototype interbody device.

FIG. 5 is a design drawing of a spinal implant in accordance with some embodiments of the present disclosure.

### DETAILED DESCRIPTION

The present disclosure may be understood more readily by reference to the following detailed description of desired embodiments included therein, the drawings, and the appended presentation which is part of the application. In the following specification and the claims which follow, reference will be made to a number of terms which shall be defined to have the following meanings.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent can be used in practice or testing of the present disclosure. All publications, patent applications, patents, and other references mentioned herein are incorporated by reference in their entirety. The materials, methods, and articles disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases that require the presence of the named ingredients/steps and permit the presence of other ingredients/steps. However, such description should be construed as also describing compositions, mixtures, or processes as “consisting of” and “consisting essentially of” the enumerated ingredients/steps, which allows the presence of only the named ingredients/steps, along with any impurities that might result therefrom, and excludes other ingredients/steps.

Unless indicated to the contrary, the numerical values in the specification should be understood to include numerical values which are the same when reduced to the same number of significant figures and numerical values which differ from the stated value by less than the experimental error of the conventional measurement technique of the type used to determine the particular value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Although the specification generally refers to PEEK, it should be understood that the compositions, devices, and processes disclosed herein may utilize other polymers. For example, the polymer may be more generically described as a polyaryletherketone. In some embodiments, the polymer is polyetherketoneketone.

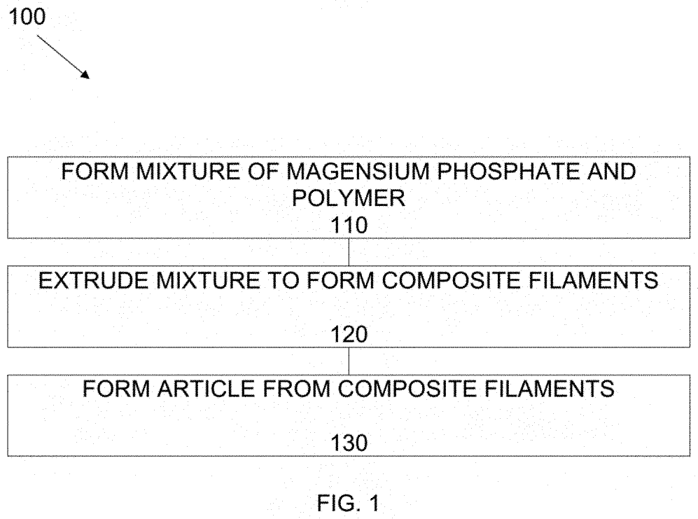

FIG. 1 illustrates a non-limiting example of a process 100 for forming an article (e.g., an interbody device) in accordance with some embodiments of the present disclosure. The process 100 includes forming a mixture of magnesium phosphate and a polymer 110, extruding the mixture to form composite filaments 120, and forming the article from the composite filaments 130.

The interbody device may be an internal bone fixation device or implant.

The interbody device may be selected from the group consisting of interbody cages, synthetic bone scaffolds, and synthetic bone plates.

One of the prime materials used in the present technology involves the FDA approved material PEEK which is used prominently in the spine industry. However, the novel formulation specifically builds upon the shortcomings of the existing technologies with an intent to resolve those.

In contrast to conventional interbody devices, the present technology intrinsically includes a bioactive material which makes the PEEK material bioactive and capable of integrating rapidly with the neighboring bone. Thus, it eliminates the need for any additional coatings.

Additionally, the compositions of the present disclosure intrinsically contain an antibacterial material which makes the PEEK interbody cages inhibit bacterial colonization and thus resists infection incidences. In addition, the antibacterial agent does not result in mutating resistant bacterial strains. Thus, it eliminates the need for systemic administration of antibiotics.

The antibacterial material may be amorphous magnesium phosphate doped with at least one transition metal. Non-limiting examples of such transition metals include silver, copper, and zinc.

The amount of transition metal(s) in the amorphous magnesium phosphate may be in the range of about 5 wt % to about 20 wt %, including from about 8 wt % to about 15 wt % and from about 6 wt % to about 12 wt %. These weight percentages are calculated with respect to the amount of magnesium content in the amorphous magnesium phosphate.

The composite material may contain from about 5 vol % to about 30 vol % of the transition metal-doped amorphous magnesium phosphate. Other non-limiting ranges include about 10 vol % to about 25 vol % and from about 15 vol % to about 20 vol %.

The composite material may contain from about 70 vol % to about 95 vol % of the matrix polymer. Other non-limiting ranges include about 75 vol % to about 90 vol % and from about 80 vol % to about 85 vol %.

Fabrication of PEEK interbody cages with intricate designs is challenging. The state-of-the-art involves the machining of bulk PEEK which involves approximately 64% material loss. An Additive Manufacturing technique known as ‘Selective Laser Sintering’ (SLS) is also used to make PEEK cages, but it also involves a significant amount of material loss. Also, sometimes the designs are not specific on the interbody cages as the SLS process is complicated by PEEK's particle morphology and size distribution. To the contrary, for the first time, the present technology involves a sustainable manufacturing technique known as Fused Filament Fabrication which does not involve any material loss and is a flexible tool to develop high-precision interbody cages with very low manufacturing cost. Thus, the cages developed are cheaper than the existing ones in the market and have high implant performance.

The present technology involves a novel class of interbody spinal fusion cages which has the potential to expedite recovery rates in patients with degenerative spinal issues. At the core of this technology lies the symbiotic combination of a well-known, FDA-approved interbody cage material (PEEK) and a novel multi-functional material known as amorphous magnesium phosphate (AMP). The result is an infection-resistant and bioactive (capable of binding to near bone rapidly) interbody device which can be the first-of-its-kind in the spine market. The device will eliminate the need for additions like Bone Morphogenic Protein which incurs excess cost and complications. The technology involves the localized release of ions to resist infections and stimulate bone formation. Importantly, for the first time, the interbody cages are developed by a sustainable additive manufacturing technique which helps in significantly reducing the manufacturing cost (by material and labor savings).

The articles, methods, and compositions of the present disclosure solve the following commercial problems:
high product cost of state-of-the-art PEEK interbody cages;
high treatment cost of antibiotic prophylaxis for inhibiting infections;
high treatment cost of using bone morphogenic protein for enhancing bioactivity; and
additional product cost due to the formation of coatings on the cages.

The innovation at-hand presents a stand-alone multi-functional interbody cage which will have a much cheaper cost as compared to the state-of-the-art cages. Furthermore, it will eliminate the additional cost incurred for the usage of antibiotics, bone morphogenic protein and implant coatings.

In addition to interbody cages, the composite materials of the present disclosure may also be useful for orthopedic scaffolds, dental implants, cranial implants, and implant accessories.

Processes for producing a patient-specific implant are also disclosed. The processes generally include image acquisition, image processing, and implant fabrication. The processes can further include implanting the implant in a patient.

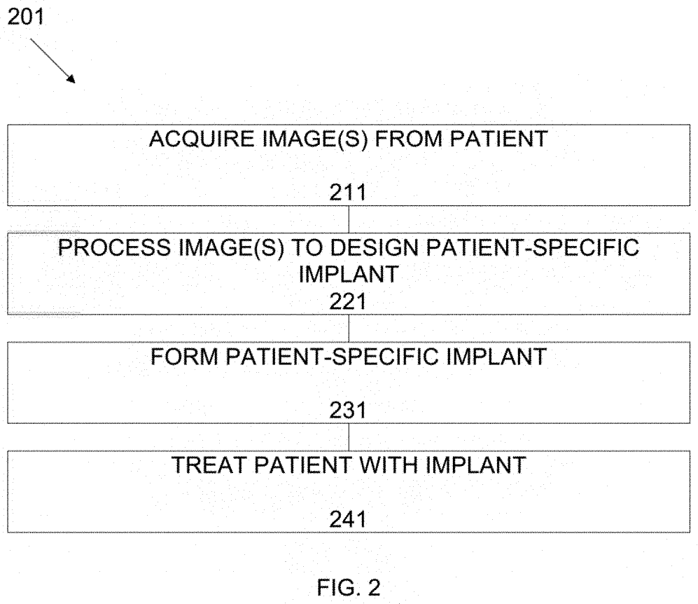

FIG. 2 illustrates a non-limiting embodiment of one such process 201. The process 201 includes acquiring at least one image from a patient 211, processing the at least one image to design a patient-specific implant 221, forming the patent-specific implant 231, and treating the patient with the implant 241.

Image acquisition generally includes capturing one or more images from a patient. The image(s) may be acquired via computed tomography (CT) scan and/or magnetic resonance imaging (MRI).

Image processing involves processing the image(s) acquired during image acquisition using a non-transitory computer-readable medium. The internal structures of the patient are analyzed to produce an implant design that will fit properly.

Implant fabrication may be performed as an additive manufacturing process (e.g., fused filament fabrication). The implant fabrication utilizes the composite material disclosed herein containing a polymer (e.g., PEEK) and a transition metal-doped amorphous magnesium phosphate.

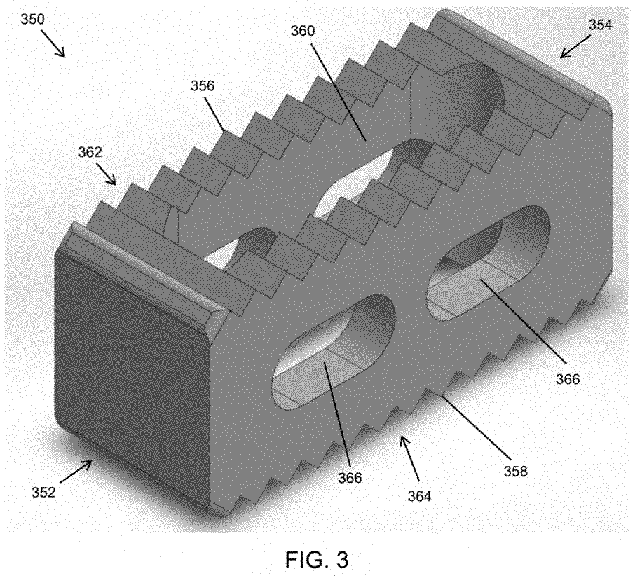

FIG. 3 is a perspective view of an interbody device 350 in accordance with some embodiments of the present disclosure. The device 350 includes a first flat or substantially flat end 352, a second, opposing flat of substantially flat end 354, a first sawtooth surface 356, a second, opposing sawtooth surface 358, an opening 360 extending through the sawtooth surfaces 356, 358, a third flat or substantially flat surface 362 extending between the sawtooth surfaces 356, 358, a fourth, opposing flat or substantially flat surface 364 extending between the sawtooth surfaces 356, 358, and a plurality of openings 366 extending through the third and fourth surfaces 362, 364. Although two openings 366 are depicted, it should be understood that there may be zero, one, three, four, or more. Also, the opening 360 may be optional or may be replaced with a plurality of openings. In some embodiments, the sawtooth surfaces 362, 364 are replaced with rounded surface elements.

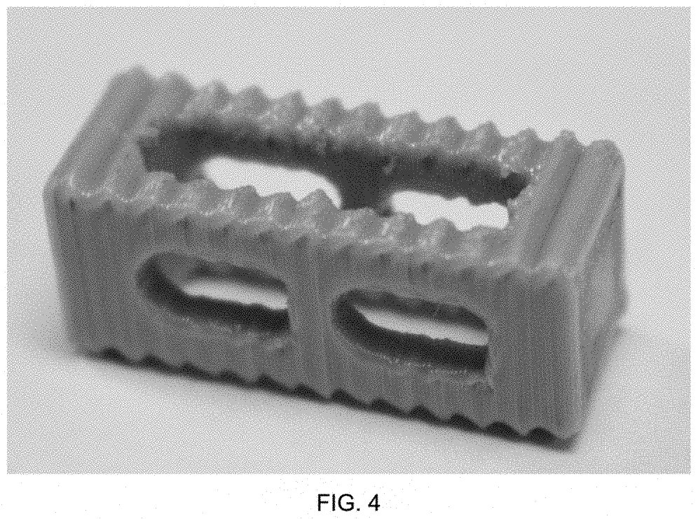

FIG. 4 is a photograph of a prototype interbody device.

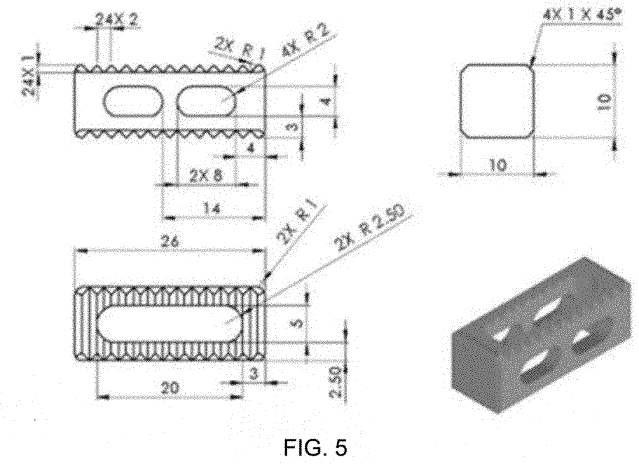

FIG. 5 is a design drawing of a spinal implant in accordance with some embodiments of the present disclosure.

Various non-limiting aspects of interbody devices, including components and shapes, are disclosed in U.S. Pat. No. 5,906,616 issued May 25, 1999; U.S. Pat. No. 8,491,653 issued Jul. 23, 2013; U.S. Pat. No. 8,673,006 issued Mar. 18, 2014; U.S. Pat. No. 8,932,360 issued Jan. 13, 2015; U.S. Pat. No. 9,364,342 issued Jun. 14, 2016; U.S. Pat. No. 10,258,481 issued Apr. 16, 2019; U.S. Pat. No. 10,470,892 issued Nov. 12, 2019; and U.S. Pat. No. 11,065,126 issued Jul. 20, 2021, the contents of which are incorporated by reference herein.

The following examples are provided to illustrate the devices and methods of the present disclosure. The examples are merely illustrative and are not intended to limit the disclosure to the materials, conditions, or process parameters set forth therein.

### EXAMPLES

Synthesis of Transition Metal-Doped AgAMP

Silver-doped AMP (AgAMP) synthesis is carried out in-house, following an ethanol-assisted co-precipitation method. 11.52 g of magnesium nitrate hexahydrate (Mg(NO₃)_{2.6}H₂O) and varying amounts of AgNO₃ 0.9, 1.1, 1.4 and 1.6 g is added to 100 ml of water and 100 ml of ethanol followed by proper stir mixing. This solution is then added rapidly, at 37° C. under constant stirring, to a solution containing 2.97 g of diammonium hydrogen phosphate ((NH₄)₂HPO₄) in 250 ml water, 45 ml ammonia (11M) and 295 ml ethanol. The resultant precipitate is collected, washed in ethanol for two times and dried in a vacuum oven overnight. The dried powder is ball milled, hand crushed and sieved to confirm the formation of fine (<75 μm particle size) powders.

Adding AgAMP in PEEK to form composite filaments.

Granulated PEEK (medical grade) is mixed with AgAMP powder with varying vol. % (20, 25, 30 vol %) for 30 minutes using an overhead stirrer. A customized filament extruder (Filastruder, Atlanta, Ga.)) and an automated filament winder (Filastruder, Atlanta, Ga.) is used to produce the 3-D printable AgAMP-PEEK filament with a constant diameter of 1.75 mm across the full length. The AgAMP-PEEK extrusion preset is selected with heater temperature in the range of 340° C.-355° C. The extruded filament is air-cooled using a fan set near the nozzle. Once the heaterreach the desired extrusion temperature, the AgAMP-PEEK mixtures is fed to the screw feeding zone through the hopper. Once a filament starts coming out of the nozzle, it is guided to the spool through the positioner to form constant diameter filaments. In the composite material, the AgAMP formed a dispersed phase in the PEEK matrix. The composite filaments contained from about 15 to about 20 vol % AgAMP and from about 80 to about 85 vol % PEEK

Fabrication of Composite Interbody Cages by 3D Printing

A fused filament fabrication 3-dimensional (3D) printer is used to fabricate the interbody cages. Before the printing process, the composite filaments is dried for 4 hours or overnight at 60° C. Subsequently, they are loaded into a high-temperature Fused Filament Fabrication 3-D printer. The filaments were fed into a 1.75 mm diameter extrusion nozzle. Simpilfy™ 3D software was used to control the printing process. A set of optimized printing temperature, printing speed, chamber temperature and printed layer height is set for the printing process. Typically, the printing temperature is set in the range of 345° C.-350° C. and the chamber temperature is set at 90° C. After the cages were fabricated, in order to enhance the mechanical properties, they are annealed for 2 hours.

In Vitro Studies

Preliminary testing was conducted to evaluate MC3T3-E1 preosteoblast cells on PEEK and a composite containing PEEK and, dispersed therein, silver doped amorphous magnesium phosphate. The results showed scant adhesion on the PEEK filaments and a thick layer adhered to the composite filaments.

In Vivo Studies

Preliminary in vivo studies were also conducted and results are provided in the appended presentation.

This written description uses examples to describe the disclosure, including the best mode, and also to enable any person skilled in the art to make and use the disclosure. Other examples that occur to those skilled in the art are intended to be within the scope of the present disclosure if they have structural elements that do not differ from the same concept, or if they include equivalent structural elements with insubstantial differences. It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims.

1. A process for forming a composite article, the process comprising:
extruding a mixture of a transition metal doped amorphous magnesium phosphate and a polymer to form extruded composite filaments; and
fabricating the article from the extruded composite filaments via additive manufacturing.

2. The process of claim 1, further comprising:
combining the transition metal doped amorphous magnesium phosphate and the polymer and mixing in a powder mixer prior to the extrusion.

3. The process of claim 1, wherein the at least one transition metal is selected from the group consisting of silver, copper, and zinc.

4. The process of claim 1, wherein the extrusion is performed with heater temperatures in the range of about 340° C. to about 350° C.

5. The process of claim 1, wherein the additive manufacturing comprises fused filament fabrication.

6. The process of claim 1, wherein the polymer is PEEK.

7. The process of claim 1, wherein the polymer is PEKK.

8. The process of claim 1, wherein the polymer is a polyaryletherketone.

9. A composition comprising a polymer and a transition metal doped amorphous magnesium phosphate.

10. The composition of claim 9, wherein the at least one transition metal is selected from the group consisting of silver, copper, and zinc.

11. The composition of claim 9, wherein the composition comprises from about 5 to about 30 vol % of the amorphous magnesium phosphate; and wherein the amorphous magnesium phosphate comprises from about 5 to about 20 wt % of transition metal(s) based on the weight of magnesium.

12. The composition of claim 9, wherein the composition comprises from about 12 to about 25 vol % of the amorphous magnesium phosphate; and wherein the amorphous magnesium phosphate comprises from about 8 to about 15 wt % of transition metal(s) based on the weight of magnesium.

13. The composition of claim 9, wherein the composition comprises from about 15 to about 20 vol % of the amorphous magnesium phosphate; and wherein the amorphous magnesium phosphate comprises from about 6 to about 12 wt % of transition metal(s) based on the weight of magnesium

14. A composite filament comprising the composition of claim 9.

15. The composite filament of claim 14, wherein the at least one transition metal is selected from the group consisting of silver, copper, and zinc.

16. The composite filament of claim 14, formed via PLA extrusion.

17. The composite filament of claim 14, wherein the polymer is PEEK.

18. The composite filament of claim 14, wherein the polymer is PEKK.

19. The composite filament of claim 14, wherein the polymer is a polyaryletherketone.

20. A process for producing an implant comprising:
acquiring at least one image from a patient;
processing the image to design a patient-specific implant; and
forming the patient-specific implant;
wherein the patient-specific implant comprises a polymer and a transition metal doped amorphous magnesium phosphate.
